(19) United States
(12) Patent Application Publication
(10) Pub. No.: US 2016/0060537 A1
(43) Pub. Date: Mar. 3, 2016

(54) RENEWABLE ENERGY STORAGE AND ZERO EMISSION POWER SYSTEM

(71) Applicant: Ztek Corporation, Woburn, MA (US)

(72) Inventor: Michael S. HSU, Lincoln, MA (US)

(21) Appl. No.: 14/931,626

(22) Filed: Nov. 3, 2015

Related U.S. Application Data
(63) Continuation-in-part of application No. 14/115,231, filed on Jan. 24, 2014.
(60) Provisional application No. 62/074,454, filed on Nov. 3, 2014.
(60) Provisional application No. 61/515,900, filed on Aug. 6, 2011.
(60) Provisional application No. 61/635,176, filed on Apr. 18, 2012.
(60) Provisional application No. 61/482,495, filed on May 4, 2011.

(51) Int. Cl. C10G 2/00; C07C 29/152; C07C 41/01; C25B 9/00; C10L 1/04; C01B 3/38; C25B 1/04; C07C 29/151; C10L 1/02

(52) U.S. Cl. CPC C10G 2/34; C10G 2/32; C10G 2/50; C07C 29/1518; C07C 29/152; C07C 41/01; C10L 1/02; C10L 1/04; C01B 3/38; C01B 3/382; C25B 1/04; C25B 9/00; C10L 2270/023; C10L 2270/026; C10L 2290/06; C10L 2290/10; C10L 2290/42; C10L 2290/38; C01B 2203/085; C01B 2203/0233; C01B 2203/0244; C01B 2203/0261; C01B 2203/86; C01B 2203/148; C01B 2203/062

(57) ABSTRACT

The invention provides an energy system comprising a fuel processor for receiving a hydrocarbon fuel and for catalytically converting the hydrocarbon fuel into a reformate, an electric heating apparatus coupled to the fuel processor for providing thermal energy to the fuel processor, an energy source coupled to the electric heating apparatus for providing power thereto, and a catalytic reactor for processing the reformate and for converting the reformate into a liquid fuel.

### RELATED APPLICATION

This application claims priority to U.S. Provisional Application No. 62/074,454, filed Nov. 3, 2014, and is a continuation-in-part of patent application U.S. Ser. No. 14/115,231, filed Jan. 24, 2014, which is a 371 application of International Application No. PCT/US2012/036640 filed May 4, 2012, claiming priority to U.S. Ser. No. 61/515,900 filed Aug. 6, 2011, U.S. Ser. No. 61/635,176 filed Apr. 18, 2012, and U.S. Ser. No. 61/482,495 filed May 4, 2011, the contents of which are hereby incorporated by reference.

### BACKGROUND OF THE INVENTION

The present application relates to a natural gas reforming plant for producing a reformate, which includes at least H₂, CO₂, and CO. More particularly, the present invention relates to a reforming plant that utilizes renewable electricity for endothermic reaction heat supply, steam generation and compression power.

The governmental efforts to curb greenhouse gas (GHG) emissions have been relying on the laws of individual countries and states for some time now. In general, most countries and states share the interim goal of reducing GHG emissions to 25% below 1990 levels by 2020, and an overall goal of reducing emissions by 80% by 2050. However, it is reported that the goals are unlikely to be met by a large margin unless suitable technologies are developed in the near future.

Current energy systems employed in an effort to produce energy efficiently typically use a steam reformer, such as a methane steam reformer, to reform a carbon based fuel into a reformate or syngas, which typically includes H₂, CO, and CO₂. The reformate can then be chemically converted into a liquid fuel, such as methanol, for subsequent use.

### SUMMARY OF THE INVENTION

The present invention is directed to an energy system that includes a fuel processor that employs an electric heating apparatus for providing thermal energy to the fuel processor during use. The use of the electric heating apparatus with the fuel processor, such as a reformer, forms in essence a hybrid reformer system. The reformer can generate a gaseous reformate, which includes H₂, CO₂, and CO, which can be further processed to remove the hydrogen from the reformate. The hydrogen can be converted, if desired, into a liquid fuel or a liquid chemical by employing a catalytic reactor. The energy system thus converts the gaseous reformate and isolated hydrogen into a liquid (GTL). The present invention thus relates to a reforming plant that utilizes electricity that is generated by an energy source, such as by a renewable energy source, for providing thermal energy to a reformer of the reformer plant to supply the necessary heat to satisfy the endothermic needs and other thermal and electrical functions of the reforming process.

The present invention is directed to an energy system comprising a fuel processor for receiving a hydrocarbon fuel and for catalytically converting the hydrocarbon fuel into a reformate, an electric heating apparatus coupled to the fuel processor for providing thermal energy to the fuel processor, a renewable energy source coupled to the electric heating apparatus for providing power thereto, and a catalytic reactor for processing the reformate and for converting the reformate into a liquid fuel or a liquid chemical. The fuel processor can be a reformer, such as a partial oxidation reformer, an autothermal reformer, a steam methane reformer, or a steam reformer.

According to one practice, the electric heating apparatus can be disposed within or external to the fuel processor . Further, the energy source is a renewable solar or wind energy source. The present invention also includes a separation unit that is coupled to the fuel processor for separating hydrogen from the reformate, and optionally a compressor coupled to the separation unit for compressing the hydrogen separated from the reformate by the separation unit, and optionally a storage element for storing the hydrogen compressed by the compressor. The reformate includes at least H₂, CO, CO₂ and H₂O.

The fuel processor can comprise a housing defining a chamber, and a plurality of reformers disposed within the housing, wherein the electric heating apparatus is mounted within the chamber for supplying thermal energy to the plurality of reformers. The electric heating apparatus has a cylindrical shape and is disposed about the plurality of reformers.

Further, the energy system can optionally include a second heating apparatus disposed within the chamber for supplying thermal energy to the plurality of reformers.

The energy system of the present invention can be constructed as a hydrogen fueling station, and if so, the system further includes a hydrogen liquefier for liquefying hydrogen produced by the fuel processor, a storage element for storing the liquefied hydrogen, a vaporizer, a gaseous H₂ holding tank, an ionic compressor, a gaseous H₂ precooler, and a hydrogen dispenser.

The input fuel for the energy system can include if desired natural gas only for startup operation, and once the system reaches a steady state operation, a portion of the H₂ generated by the fuel processor can be recycled to replace the natural gas as an input fuel, so as to form a zero emission system. Also, the energy system generates CO₂ and utilizes the CO₂, which can be pressurized or stored in a liquefied state, to fulfill the renewable function in energy or power system operation. The energy system of the present invention can practice steam recycle to enhance the thermal efficiency of a co-production process, which is to recycle steam production and utilization between power production and reforming process. The energy system converts renewable raw electricity to a form suitable for storage to become dispatchable as an on-demand, regulated commercial power, such as on-peak or average renewable power rating.

According to the present invention, the fuel processor produces a reformate having at least H₂, CO₂, and CO, and is further processed into two separate streams, including a stream of relatively pure H₂ and a stream of CO₂, where the stream of H₂ is employed for zero emission power generation and the stream of CO₂ is applied for CO₂ chemical and commercial usage.

The present invention is also directed to a method for producing a liquid fuel, comprising catalytically converting a hydrocarbon fuel into a reformate with a fuel processor, providing thermal energy to the fuel processor by an electric heating apparatus, providing power to the electric heating apparatus by an energy source, and processing the reformate and converting the reformate into a liquid fuel with a catalytic reactor. The electric heating apparatus is disposed within or external to the fuel processor.

The present invention is also directed to an energy system, comprising an energy source for providing electricity, an electrolyzer positioned so as to receive the electricity from the energy source and water from a water source, for converting the water into a hydrogen stream and an oxygen stream, a combustor for combusting a fuel received from a fuel source and the oxygen stream received from the electrolyzer and for generating an exhaust, and a catalytic reactor for processing the exhaust from the combustor and the hydrogen stream from the electrolyzer for producing a liquid fuel or a liquid chemical. The energy source is a renewable solar energy source or a renewable wind energy source. The liquid fuel or the liquid chemical can be used for electric energy storage with no strict constraints in quantity or storage shelf life. Further, the liquid chemical can be used in the form of plastic from methanol to olefins plastics by employing an MTO process with zeolite H-SAPO-34, and which can be disposed as HC solid waste without releasing CO₂ to the atmosphere.

Further, with regard to renewable energy storage technology, electrical storage systems can be classified into three general categories, namely, a power storage device category, an energy storage system category, and an energy conversion system category.

The power storage device category contemplates the use of a power storage device that has the ability for fast charge and discharge, but with limited capacity for holding the energy. These storage devices are known in the art, and include for examples commercially available battery technology. The energy storage system provides increased but still finite capacity, and can include for example flow batteries. The third category involves the integration of chemical/mechanical/electrical components of electrochemical devices, such as electrolytic cells and fuel cells of unlimited capacity with the storage media for regeneration or other commercial applications. The media consist of hydrogen, hydrocarbon in gaseous or liquid form.

The foregoing energy conversion system is just now being explored since the demand for energy continues to increase. In the transportation industry where the use of electric vehicles continues to increases, the distance that the vehicle can travel on a single charge has been found to be marginal and thus inadequate for modern day driving needs. In the renewable energy industry, the ability to adequately store energy produced by wind power is lacking.

The present invention is intended to address in part at least some of the foregoing deficiencies. The use of a fuel processor, for example, a reformer, that employs an electrical heating apparatus to provide thermal energy to the reformer can be used to generate hydrogen for subsequent use, such as for example by a fuel cell, a fuel cell vehicle, power generating devices and the like. The reformer systems thus function when renewable electricity is supplied thereto as an energy efficient storage system of renewable energy of, for example, wind and solar renewable resources.

The present invention also is directed to a clean energy system that utilizes renewable energy sources to produce renewable hydrogen that serves many clean energy functions. These functions include the storage of surplus renewable energies in the form of renewable hydrogen, which can be used as fuel for power sources, such as fuel cells and fuel cell vehicles, and as fuel for clean energy power generation for zero emission battery cars and stationary power needs. The renewable hydrogen can be made storable by liquefying the hydrogen and then if needed storing the liquefied hydrogen. The renewable hydrogen can be made storable by reacting with CO₂ in a methanation process to form a renewable gaseous Fuel, such as CH₄ or CH₃OH.

Further, any CO produced during the reformation process can be compressed to a liquid state for compact storage and/or supplied as a gas for further use.

The present invention converts renewable raw electricity or perishable energy to a form suitable for storage to later become dispatchable or employable, on demand, regulated commercial power, such as on peak or average renewable power rating.

### BRIEF DESCRIPTION OF THE DRAWINGS

The foregoing and other objects, features and advantages of the invention will be apparent from the following description and apparent from the accompanying drawings, in which like reference characters refer to the same parts throughout the different views. The drawings illustrate principles of the invention.

FIG. 1 is a block diagram illustrating clean technology applications including the embodiments provided in the present application.

FIG. 2 is a block diagram of an exemplary clean energy system or zero emission energy system (ZEES) with CO₂ waste utilization in an illustrative embodiment according to the teachings of the present invention.

FIG. 3 is an overview of a zero emission energy system (ZEES) provided according to the teachings of the present invention.

FIG. 4 shows the structure of an exemplary reformer that may be employed in the zero emission energy system (ZEES) depicted in FIG. 3.

FIG. 5 shows the structure of an exemplary energy reactor that may be employed in the zero emission energy system (ZEES) depicted in FIG. 3.

FIG. 6 depicts another embodiment of an exemplary clean energy system according to the teachings of the present invention supporting solar power storage of FIG. 7 and CO2-EOR for oil recovery of FIG. 8.

FIG. 7 is a block diagram of an exemplary energy system supporting Photovoltaic (PV) solar power storage for local grid use in an illustrative embodiment.

FIG. 8 shows an exemplary CO₂-Enhanced Oil Recovery (EOR) system provided in an exemplary embodiment.

FIG. 9 is a schematic representation of another embodiment of the energy system of the present invention.

FIG. 10 is a schematic cross-sectional view of an exemplary reformer employed in the energy system of FIG. 9 that includes an internally mounted electrical heating unit.

FIG. 11 is a schematic representation of still another embodiment of the energy system of the present invention.

FIG. 12 is a schematic representation of yet another embodiment of the energy system of the present invention.

### DETAILED DESCRIPTION OF THE INVENTION

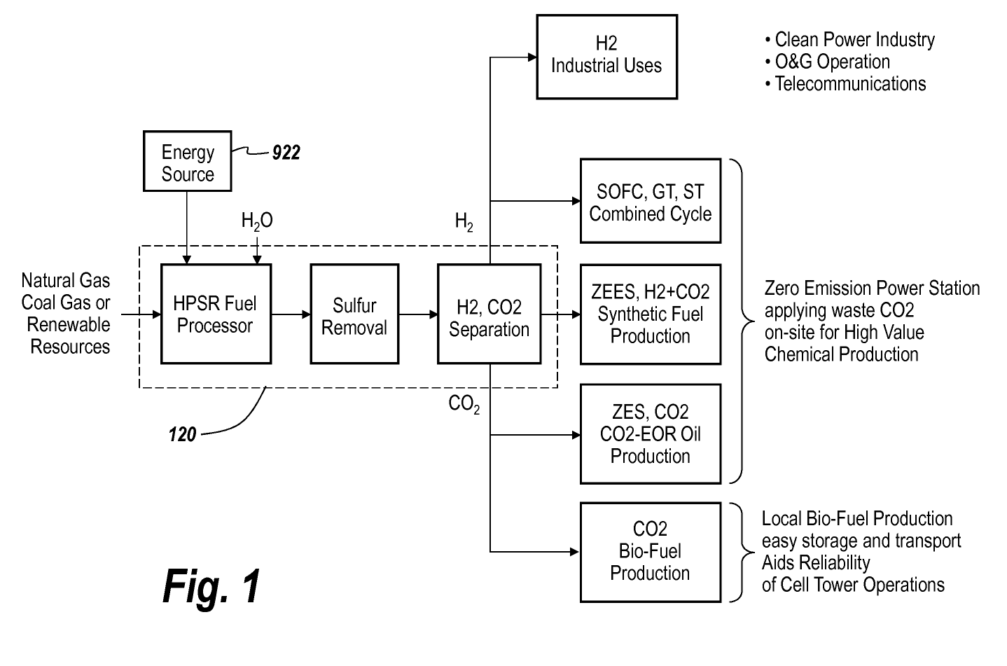

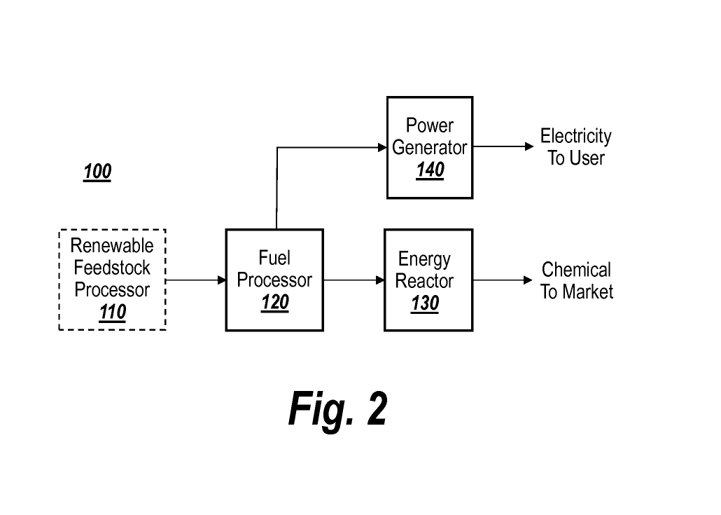

FIGS. 1 and 2 are schematic block diagrams illustrating an energy system 100 according to the teachings of the present invention. The illustrated energy system 100 can include a fuel processor 120, an energy or catalytic reactor 130, and a power generator 140. The illustrated energy system converts a carbon based fuel product into a more refined or pure fuel for subsequent use or storage.

The illustrated fuel processor 120 can receive a carbon based fuel, such as natural gas, coal gas or a renewable gas, and process the gas to form a reformate, which can include H₂, CO, CO₂ and/or a mixture of H₂,CO and CO₂. The fuel processor can include any suitable type of reformer having any suitable structure or configuration, such as an autothermal reformer, a partial oxidation reformer, or a steam reformer (e.g., a steam methane reformer). As shown the reformer processes or reforms the fuel, and the processed fuel can pass through a sulfur removal apparatus for removing sulfur from the processed fuel, to form the reformate. The hydrogen in the reformate can then be separated from the remaining reformate gases by known techniques. For example, a series of gas or temperature shift reactions or water processes can be employed to separate the hydrogen. The reformate may be processed with a water shift process to have different percentages of CO vs. CO₂ with a variation range between about 0% and about 20%. Alternatively a pressure swing adsorption device can be employed to perform the same. The reformate of the fuel processor may be processed with a pressure swing adsorption process to form two flow streams, namely, a high purity H₂ steam and a high carbon concentration stream (CO, CO₂). The CO₂ may be processed with the water shift process to the maximum level of 20%, and processed through the pressure swing adsorption to result in a concentrated CO₂ stream. The concentrated CO₂ stream may be applied for Enhanced Oil Recovery (CO₂-EOR) on-site at oil wells as well as many other types of commercial applications.

The fuel processor of the present invention can be a reformer, and more particularly can be a hybrid reforming system involving electric energy input obtained from a power source, such as a renewable power source such as solar (e.g., photovoltaic (PV) panels), wind power or tidal wave power, to meet the endothermic energy needs of the reformer and the reformate compression process. The energy system may be a hybrid system involving electric energy input obtained from the PV panels, wind and tidal wave to support the energy demands for the system, as well as to provide remedy for the intermittency of these power sources with storage capability in the form of a liquid fuel or a liquid chemical. As illustrated, an energy source 922 can be provided so as to provide electricity to the fuel processor. The energy source can employ renewable and/or nonrenewable energy sources as desired.

As shown in FIG. 1, the produced H₂ may be used as a fuel source for a solid oxide fuel cell (SOFC) system. The SOFC also has an advantage in that its clean exhaust remains at a high temperature, which is suitable to drive a traditional system for additional power generation. The overall exhaust remains clean while the power output or the system efficiency doubles. The SOFC system may be integrated with heating, ventilation and air conditioning (HVAC) systems to perform functions for human comfort using the hot exhaust from the SOFC. When energy costs rise, the SOFC-HVAC system may be made available to serve families and communities with a single system performing all necessary power generation functions.

According to one practice of the present invention, the reformate may be applied for power generation to other types of fuel cells, including molten carbonate fuel cells (MCFC), proton exchange membrane fuel cells (PEMFC), phosphoric acid fuel cells (PAFC) and alkaline fuel cells (AFC). The SOFC can be provided in different arrangements, including as a stand alone fuel cell or a plurality of SOFCs, as part of a hybrid fuel cell system composed of a solid oxide fuel cell and gas turbine unit, or as part of a hybrid system composed of a solid oxide fuel cell and a steam turbine unit. The reformate may be applied for power generation in one of the following generators including an internal combustion engine, gas turbine, or steam turbine.

Further, the H₂, CO and CO₂ produced from the fuel processor may also be processed by an energy or catalytic reactor 130, FIG. 2, into methanol (CH₃OH) in liquid form with a methanol synthesis catalyst. The methanol may be further processed in the energy reactor into DME (CH3OCH3) in liquid form with a suitable catalyst. The DME may be further processed in the catalytic energy reactor into gasoline in liquid form with another suitable catalyst. The mixture of H₂, CO₂ and CO derived from the renewable feedstock may be carried out for producing jet fuel, propane, diesel or heavy liquid fuel production due to high concentrations of CO₂ or CO in the syngas.

According to the teachings of the present invention, the energy system 100 may be operated to maximize the use of carbon for the production of liquid bio-fuels, including methanol, ethanol, propanol and butanol, as well as liquid synfuels, including DME, gasoline, propane, butane, jet fuel and diesel. The energy or catalytic reactor utilizes chemical-catalysts or bio-catalysts. The energy reactor may be a chemical-catalyst bed, such as a fixed bed, structured bed, slurry bed or microchannel with integrated heat exchanger. The energy reactor may employ single functional catalysts, bifunctional catalysts or multi-functional catalysts to achieve improved performances. The energy reactor may employ bio-catalysts including varieties of yeasts, bacteria and enzymes.

A Zero Emission System (ZES) can also be employed to make use of CO₂ from the energy system 100, in which the hydrogen is fully utilized while the CO₂ is exhausted to the air as unwanted emissions. CO₂ is produced in the system in a concentrated stream for subsequent collection. In one embodiment, the CO₂ gas may be used for injection into an oil field or well to enhance oil production. The CO₂ gas may be compressed for CO₂-EOR (Enhanced Oil Recovery). This may provide opportunities for all small and medium field owners to enjoy the benefits of CO₂ in their wells, in areas of absence of natural CO₂ supply services typically available only to large field owners. The well-known technique of horizontal drilling when coupled with CO₂ injection may provide a deeper and wider reach into the earth with further benefits in thorough sweeping of the sand grains by CO₂ action for enhanced oil production. The carbon dioxide produced by the energy system 100 can also be employed to produce selected bio-fuels.

An embodiment of the present invention also provides a zero emission energy system (ZEES) for eliminating the CO₂ emissions from the traditional power plants and introducing new sources of liquid fuel for transportation. The embodiment utilizes chemical principles for concurrent power generation and energy conversion. The fuel stock is first treated before the power generation to assure clean emissions. The hydrocarbon fuel, either from a petro-source or from a bio-source, is processed following the steam methane reformer reaction into H₂, CO, CO₂ and H₂O, and collectively constitute the reformate. The reformate may be separated into two gas streams, namely, a H₂ stream and a carbon containing stream. The H₂ stream is primarily utilized for power generation that emits only H₂O (e.g., water molecules). The carbon stream when combined with the proper amount of H₂ from the H₂ stream may be catalytically reacted to form liquid fuels, such as methanol (CH3OH). Other liquid fuel species may be further derived with suitable catalysts. The energy system may provide solutions to simultaneously cure the problems in the current energy industry: petro-fuel shortage and global warming due to greenhouse gas (GHG) emissions. In the energy system, the clean power is generated and the greenhouse gas CO₂ is retained and applied for the production of high valued fuels.

The ZEES provides the energy industry the following benefits: ZEES provides storage solutions to the Solar Electric projects in liquid chemicals; ZEES provides sweeping solution to power generation of emissions issues; ZEES promotes global oil production to relief international political worries; ZEES as GTL facilitates the natural gas move in convenient liquid forms; ZEES as BTL facilitates the renewable bio-feedstock to become commercial energy source; ZEES as a chemical reactor to process petro fuel or biofuel with equal effectiveness; ZEES hybridizing with renewable electricity for storage better serve the future Smart Grid; ZEES thus provides an unhurried transition to better fuel choices.

In an embodiment, the energy system of the present invention may be configured to use renewable feedstock that includes municipal solid waste, municipal sewage, farm animal waste, biomass and woody biomass with a feedstock processor. The municipal sewage and farm animal waste may be first processed through a digester to yield biogas, which includes methane CH₄, CO₂ and CO. The municipal solid waste, farm biomass, woody biomass may be first processed through a gasifier to yield syngas which includes methane CH₄, CO₂ and CO. According to the present invention, the feedstock processor may be a syngas generator (e.g., a volatizer), including a thermal driven, plasma or microwave driven gasifier.

FIG. 2 is a schematic block diagram of an exemplary energy system or zero emission energy system (ZEES) with CO₂ waste utilization according to the teachings of the present invention. The ZEES is uniquely qualified for distribution or installation near energy source, market place, and with product shippable for off-site consumption. As used herein, the zero emission energy system refers to an energy system that processes an input feedstock, such as a natural gas or a renewable gas, and produces power with substantially zero emissions of CO and CO₂ gases to the atmosphere. The CO and CO₂ gases produced by processing the input feedstock can be utilized to produce a liquid fuel. The CO and CO₂ gases produced by processing the input feedstock can also be injected into a wellhead to enhance oil recovery.

The energy system 100 may include a fuel processor 120, such as a reformer, an energy catalytic reactor 130, and a power generator 140. The fuel processor 120 can be a reformer, such as a steam reformer, that converts a renewable feedstock, such as methane rich gas, into a reformate including H₂, CO and CO₂, which in turn can be introduced to a fuel cell, such as a solid oxide fuel cell (SOFC). Within the steam reformer, any sulfur present in the reactant may be removed and the reactant pressure can be adjusted. The water is treated for deionization to prevent corrosion to the equipment in the system and to precondition the reactant for clean emissions. The reformate outputted by the fuel processor 120 may be processed into two separate streams, a H₂ rich stream and the carbon containing CO and CO₂ stream.

The energy or catalytic reactor 130 converts H₂ with CO and CO₂ into a liquid form of chemical under catalytic reaction according to well-known techniques. Methanol has been recognized as a fundamental liquid chemical derived from syngas or biogas with a commercially available methanol catalyst reactor. Methanol may be further processed into various levels of alcohols, such as ethanol, pentanol, butanol and various commercial fuels such as gasoline, jet fuel and diesel. Gaseous fuel species are difficult to transport or distribute from production plants to the market place. Thus, it is desirable to be able to convert it into liquid form for storage as well as for shipping to market. It also gains the advantage of being used as the fuel for transportation, or vehicle fuels.

The power generator 140 may use the H₂ rich stream derived from the reformer 120 to generate power, a byproduct of which is water. It is a desirable and efficient mode for power generation. The traditional power generators, such as internal combustion engines, gas turbines and steam turbines, may also be powered by the H₂ gas stream. Fuel cells are a suitable generator class on H₂ fuel. The energy system 100 transforms various input gases, such as natural gas, syngas or biogas, into hydrogen rich gas that may be used by the electrochemical reaction of the fuel cell. The Gas Turbine (GT) portion of the hybrid

SOFC-GT is capable of reusing the SOFC exhaust to boost the overall efficiency of the combined cycle system to about 70%. The zero emission energy system (ZEES) may further be applied in renewable applications when an optional renewable feedstock processor 110 is added prior to the fuel processor 120.

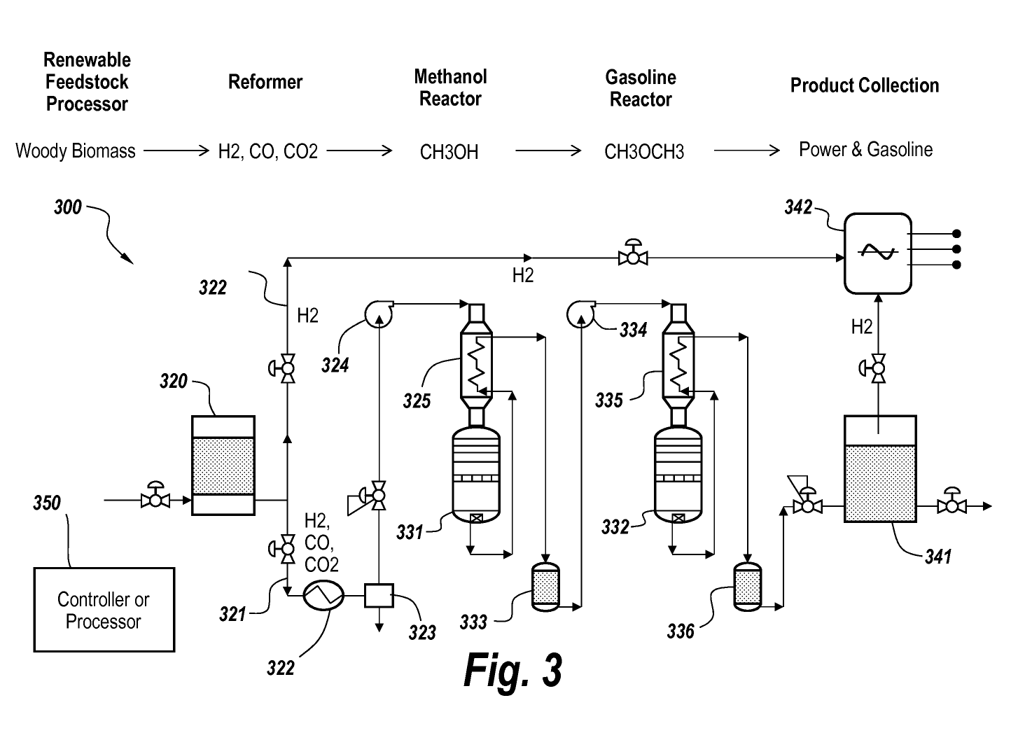

FIG. 3 is an overview of a zero emission energy system 300 provided in an exemplary embodiment. The clean energy system 300 may include a fuel processor 320, energy reactors 331 and 332 and a power generator 342. The fuel processor 320 may be a reformer system that catalytically breakdown the CH₄ component in the fuel source, such as natural gas, biogas or syngas, into H₂, CO, CO₂ and H₂O species joining the output flow and collectively is called the reformate. A power generation branch 322 feeds a fuel reactant for power generation with fuel cells or other traditional generators or engine forming the power generator 342. The energy or catalytic reactors 331 and 332 are, for example, gas to liquid reactors fed by the liquid chemical production branch 321 of the reformate.

The fuel processor 320 may be a partial oxidation, autothermal or steam methane reformer. The steam methane reformer provides the best performance for pure hydrogen production, if steam supply is unrestricted. A water shift processor following the reformer may be controlled by a processor (not shown) to have a different percentage of CO vs. CO₂ in a range from about 0% to about 20%.

The above reactions are expressed as:

Steam Methane Reforming CH₄+H₂O→3H₂+CO

Water Gas Shift Reaction CO+H₂O→CO₂+H₂

The reformate may be further processed in a pressure swing adsorption process into two streams of flows: 1) high purity H₂ steam; 2) high concentration of carbon (CO, CO₂) contents.

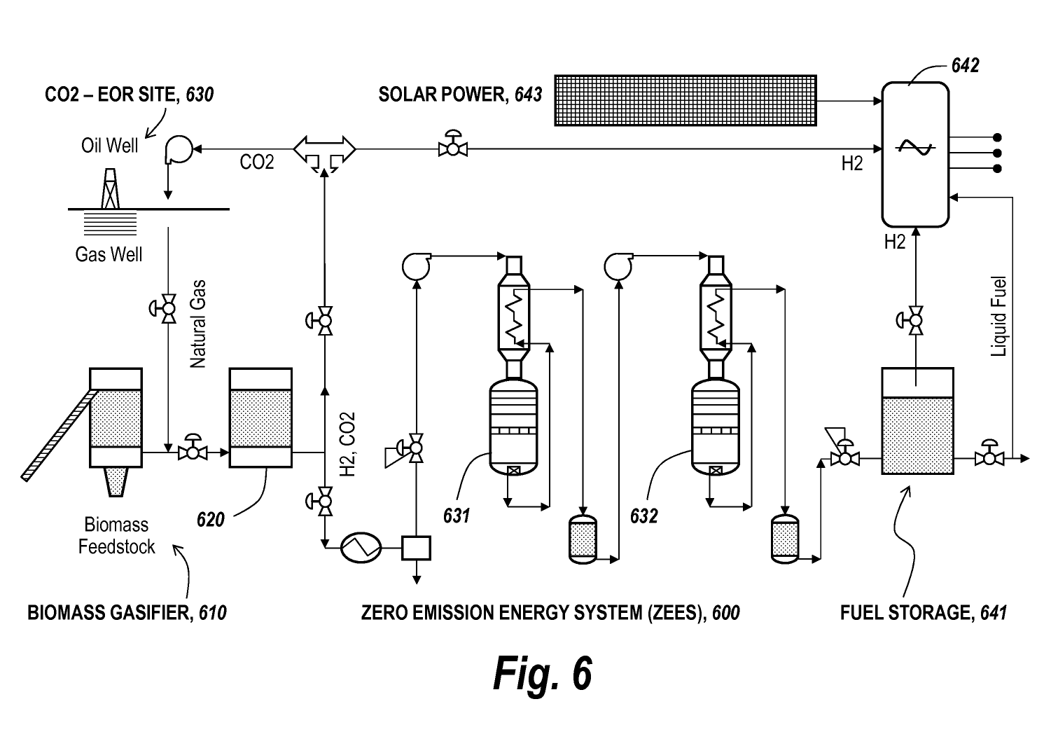

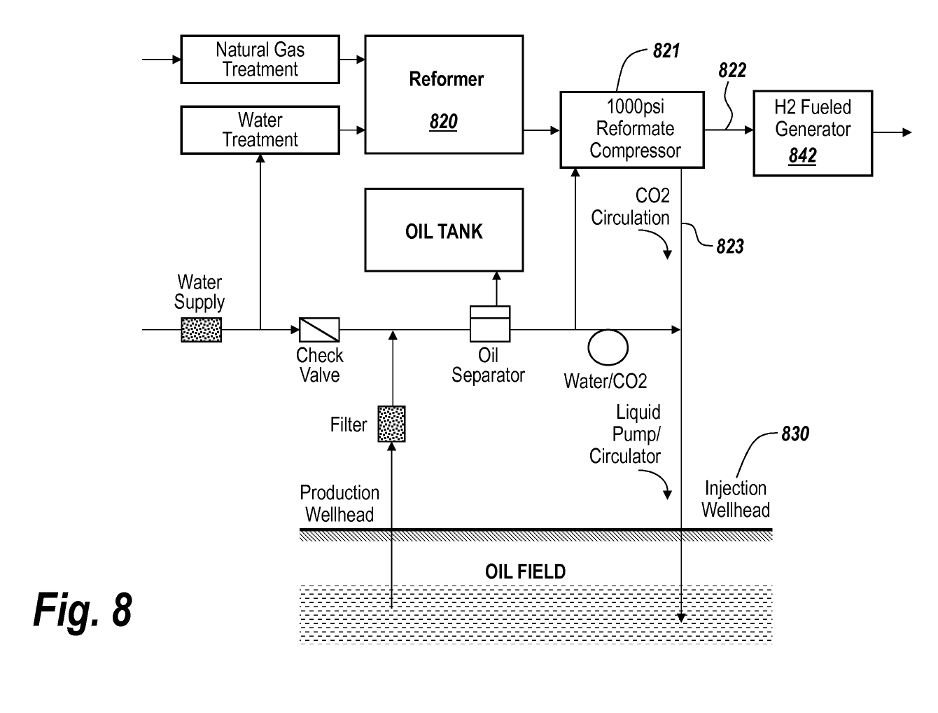

When the reformate is processed in the water shift processor to the maximum level of 20% with low or trace amounts of CO, it may be further processed through the pressure swing adsorption process to yield a concentrated CO₂ stream. The concentrated CO₂ stream may be applied for Enhanced Oil Recovery (CO₂-EOR) on-site at oil wells, as shown in FIG. 6 and FIG. 8.

The liquid chemical production branch 321 carries a mixture of H₂, CO and CO₂ which is introduced into the energy reactor 331 via a heat exchanger 322, a condenser 323, a compressor 324 and a heat exchanger 325 for the production of liquid methanol (CH3OH) with a methanol synthesis catalyst. The condenser 323 may extract water from the reformate and the compressor 324 compresses the reformate before it enters the heat exchanger 325. The methanol may be further introduced into a second energy reactor 332 via a heat exchanger 333, a compressor 334 and a heat exchanger 335 for production of DME (CH3OCH3) in liquid form with a suitable catalyst. The DME as produced may be further processed in a catalytic energy reactor into common gasoline in liquid form with suitable catalyst. The DME or gasoline may be sent to the storage 341 via a heat exchange 336.

The above reactions can be expressed as:

Methanol Synthesis 2H₂+CO→CH₄O

Dimethyl Ether (DME) Synthesis 2CH₄O→CH₃OCH₃+H₂O

The zero emission energy system 300 may be operated to maximize the use of carbon for the production of liquid bio-fuels, including methanol, ethanol, propanol and butanol, as well as liquid synfuels, consisting of DME, gasoline, propane, butane, jet fuel and diesel. The liquid fuel or chemicals can be used in the form of plastic from methanol to olefins plastics by an MTO Process employing for example zeolite H-SAPO-34 that can be disposed as HC solid waste without releasing CO₂ to the atmosphere.

The energy reactor 331 or 332 utilizes chemical-catalysts or bio-catalysts respectively. The energy reactor 331 or 332 employs chemical-catalyst bed in one of the various possible configurations including a fixed bed, structured bed, slurry bed and microchannel with integrated heat exchanger. The microchannel reactor may be of a cylindrical configuration. The energy reactor 331 or 332 may employ single functional catalysts, bifunctional catalysts or multi-functional catalysts to achieve improved performances. The bifunctional or multi-functional catalysts are formed by thorough mixing of basic compounds of individual catalysts, resulting in near instant chained-reactions without unnecessary time lapse. This innovation helps to reduce the size and the number of the reactors, thus leading to cost reduction in system construction. The energy reactor 331 or 332 may also employ bio-catalysts consisting of varieties of yeasts, bacteria and enzymes.

The H₂ stream 322 may be applied for power generation by a power generator 342 with zero CO₂ emission by employing fuel cells or other traditional power generators. The fuel cell electric generators may be selected from one of the types from solid oxide fuel cell (SOFC), molten carbonate fuel cell (MCFC), proton exchange membrane fuel cell (PEMFC), phosphoric acid fuel cell (PAFC) and alkaline fuel cell (AFC). The high performance SOFC has three configurations: a solid oxide fuel cell; a hybrid system composed of a solid oxide fuel cell and gas turbine unit; a hybrid system composed of a solid oxide fuel cell and steam turbine unit. The traditional power generation consists of one of the following generators selected from internal combustion engine, gas turbine and steam turbine.

Various pump and valves can be employed throughout the system to help regulate or control the flow of the various fluids. These components can be controlled by a controller or processor 350.

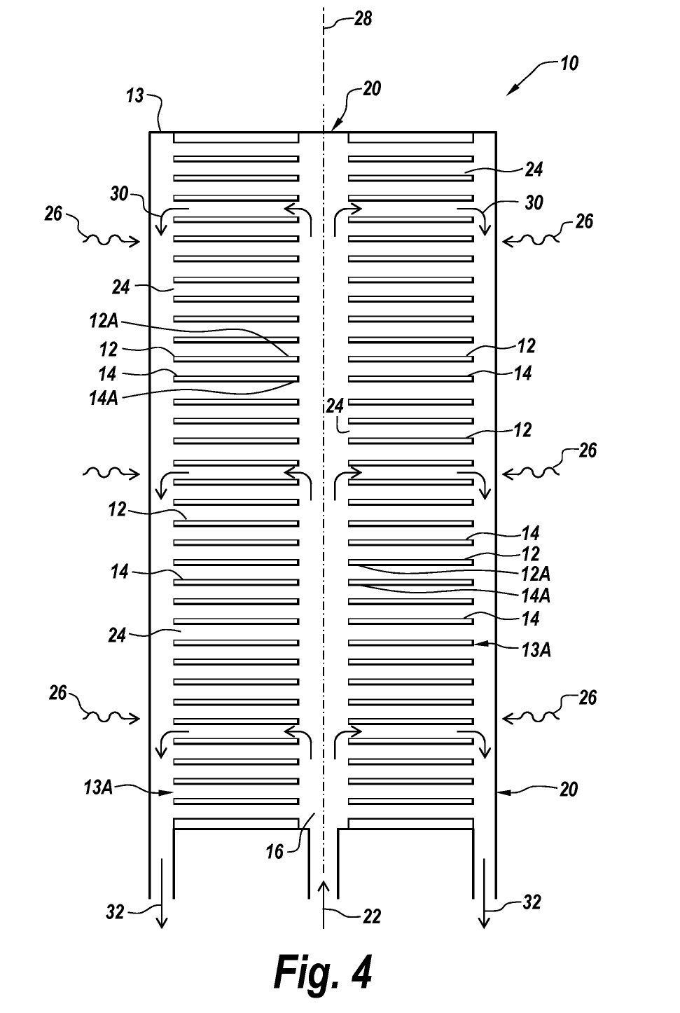

FIG. 4 shows the structure of an exemplary fuel processor suitable for use in the zero emission energy system (ZEES) depicted in FIGS. 1-3. The fuel processor can be a reformer, as noted above. The illustrated reformer 10 may include a stack of thermally conducting plates interspersed with catalyst plates and provided with internal or external manifolds for reactants. The catalyst plate is in intimate thermal contact with the conducting plates so that its temperature closely tracks the temperature of the thermally conducting plate, which may be designed to attain a near isothermal state in-plane to the plate. One or more catalysts may be used, distributed along the flow direction, in-plane to the thermally conducting plate, in a variety of optional embodiments. The reformer may be operated as a steam reformer or as a partial oxidation reformer. When operated as a steam reformer, thermal energy for the (endothermic) steam reforming reaction is provided externally by radiation and/or conduction to the thermally conducting plates. This produces carbon monoxide, hydrogen, steam and carbon dioxide. When operated as a partial oxidation reformer, a fraction of the natural gas is oxidized assisted by the presence of a combustion catalyst and reforming catalyst. This produces carbon monoxide, hydrogen, steam and carbon dioxide. Because of the intimate thermal contact between the catalyst plate and the conducting plates, no excessive temperature may develop within the stack assembly. Details of the plate design may be varied to accommodate a variety of manifolding embodiments providing one or more inlets and exit ports for introducing, pre-heating and exhaust the reactants.

The reformer 10 includes a number of thermally conductive plates 12 and reforming plates 14 that are alternately stacked together to form a stacked reforming structure 13 that extends along axis 28. The reformer includes a fluid conduit 16 that is in fluid communication with the inner portions 12A, 14A of the plates 12, 14. The reformer 10 is preferably housed within a gas-tight enclosure or housing 20. The illustrated reformer may be used to perform both steam and oxidation reforming. The heat necessary for the reforming process may be supplied internally by partial oxidation of hydrocarbon fuel or supplied externally by a remote heat source, as shown by wavy lines 26, to the reformer 10 by radiation, conduction or convection. The thermal energy can be provided by any suitable external heat source, including for example from a combustor or from a fuel cell, such as a SOFC.

The reactant to be reformed by the reformer 10 is introduced into the apparatus through the axial fluid manifold 16. The reactant preferably comprises a mixture of a hydrocarbon fuel and a reforming agent, such as air, oxygen, water or CO₂,that are premixed either prior to introduction to the manifold 16 or within the reformer. The illustrated reformer 10 includes at least one manifold that delivers a fuel/reforming agent mixture to the reformer, rather than provide separate input manifolds for each gas constituent. The introduction of a premixed reactants to the reformer 10 provides for a relatively simple design.

The reactant mixture 22 is introduced to the manifold 16 by any appropriate means, such as by fluid conduits. The mixture 22 enters the inner portions of the reformer through reactant passages 24 that are formed between the adjacent conductive plates 12 and reforming plates 14. The passages may comprise any surface indentation or protrusions, which may be formed by embossing, and which constitutes a substantially continuous fluid passage that extends from the manifold 16 to the outer peripheral surface 13A of the stacked reforming structure 13. The passages may also be formed by utilizing conductive or reforming plates that are made of a porous material or have a power reformer catalyst material coated or formed thereon, thus allowing the reactant to pass through the reformer.

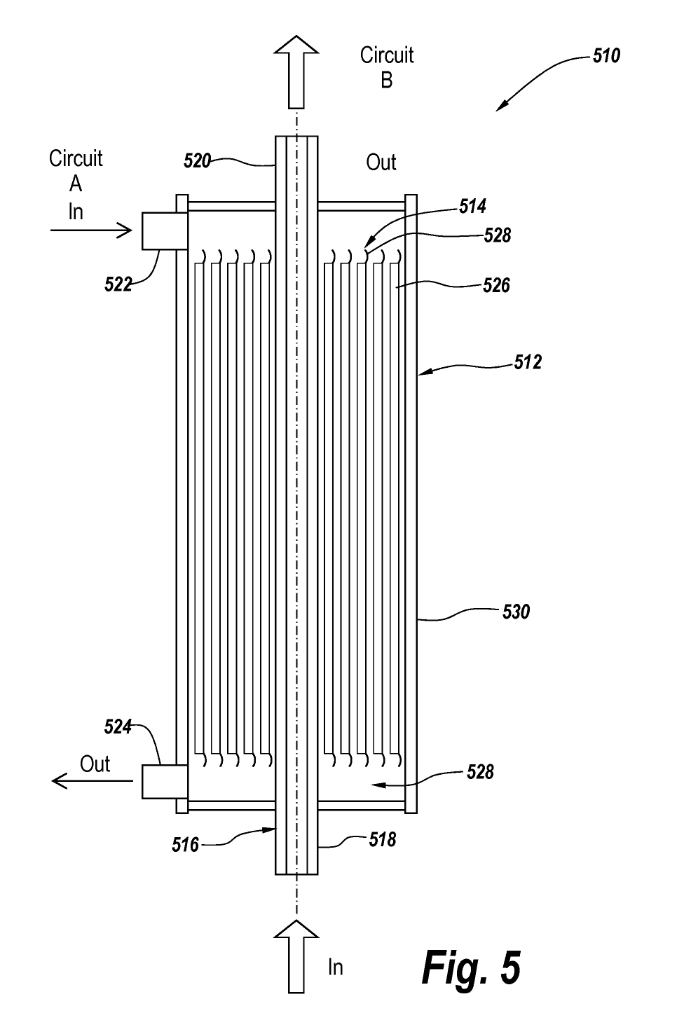

FIG. 5 shows the structure of an exemplary energy or catalytic reactor suitable for use in the zero emission energy system (ZEES) of the present invention, such as the system of FIG. 3. The reactor 510 may be a cylindrical catalytic reactor or a cylindrical micro-channel (CMC) reactor that has a cylindrical micro-channel (CMC) structure to incorporate catalyst to be used as a catalytic reactor. The reactor 510 may include a housing 512 defining a chamber 528 that has an inlet 522 and an outlet 524. The housing can have any selected shape or size, and preferably has a cylindrical shape. A bundle element 514 is mounted within the chamber 528. The bundle element can include a conduit and a multi-sheet layer 526 which can be wrapped around the conduit. The multi-sheet layer 526 can comprise at least two sheets which define a confined flow volume. The bundle element 514 can also be constructed as a series of tubes. The bundle element 514 can include a conduit 516 that passes through the housing 512 and extends between an inlet 518 and an outlet 520. The inlet and outlet do not communicate directly with the chamber 528.

In an embodiment, the bundle element includes double layers. The space formed between the double layers is designated as the B-side (circuit B). The space formed between the rolled double layers is designated as the A-side (circuit A). The A-side allows straight flow path along the axis of the reactor with an inflow port 522 and outflow port 524. The B-side presents a spiral flow path between the double layers with an inflow port 518 and outflow port 520. A round screen or perforated plate is placed and secured at the one end (bottom) of the cylinder, which allows the loading of catalyst of proper size to fill the A-side of the reactor when positioned upright for operation. A removable round screen or perforated plate may be placed at the other end (top). The B-side is used as the path for the thermal fluid to flow through for temperature regulation, heating or cooling.

The space between the “double layers” and the space between the “spirally rolled double layers” may be supported by dimples formed in the sheets before welding and rolling. The space may be controlled in millimeter sizes to provide excellent heat transfer capability between the medium on the A-side through the metal layer to the B-side. The rolled configuration presents large surface area to be packaged in small cylindrical volume.

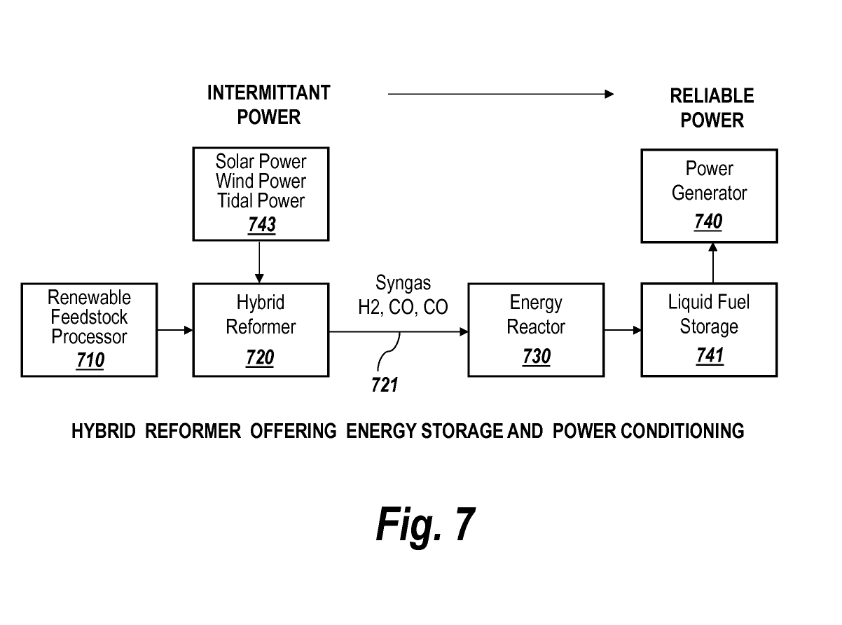

FIG. 6 depicts another embodiment of an exemplary zero emission energy system 600 according to the teachings of the present invention supporting solar power storage of FIG. 7 and CO₂-EOR for oil recovery of FIG. 8. The zero emission energy system 600 may include a fuel processor 620, energy reactors 631 and 632, a fuel storage element 641 and a power generator 642, which are substantially the same elements as described above with respect to FIG. 3. The array of solar cells or solar panel 643 can be used to generate electricity for subsequent use.

The zero emission energy system 600 may be applied to use renewable feedstock that includes municipal solid waste (MSW), municipal sewage, farm animal waste, biomass and woody biomass with a feedstock processor. The municipal sewage and farm animal waste may be first processed through a digester to yield biogas, which includes methane CH4, CO₂ and CO. The municipal solid waste, farm biomass, woody biomass may be processed through a gasifier 610 to yield syngas which includes methane CH₄, CO₂ and CO.

The feedstock processor may be a syngas generator, selected from a thermal driven, plasma or microwave driven gasifier. A desirable syngas generator classified as a volatilizer 610 provides syngas of CH₄ rich having heating value exceeding 500 Btu/ft3, other than a gasifier of a common choice providing syngas of H₂ rich with heating value typical of 300 Btu/ft3. The renewable feedstock includes forest waste, community (municipality) waste, coal of solid phase and even waste of liquid phase or sludge which passing through the volatilizer yields syngas and other secondary species including variable amount of biochar or charcoal as the solid residual.

The volatilizer 610 may process the biomass under physical step without undergoing combustion, rather undergoing heating by external sources. The external heating source may be derived from the recycled portion of the syngas, which provides clean burn high temperature source of heat above 1000° C. The external heating source may also derived from the high temperature waste stream, 800° C. to 1000° C., of the SOFC power generator or the high temperature waste stream, 600° C. to 800° C., of the hybrid power generator.

The biochar left behind in volitilizer 610 has commercial value as the activated charcoal for commercial use and may claim for carbon credits and renewable energy credits.

The clean energy system 600 is a zero emission energy system when carbon containing stream with matched amount of H₂ is used for liquid fuel production and H₂ alone is used for power generation. The system when applied to use renewable feedstock constitutes negative CO₂ footprints thus gaining double carbon credits.

FIG. 7 is a block diagram of another embodiment of the zero emission energy system of the present invention that supports the use of photovoltaic (PV) or solar power storage for local grid use. The energy system is provided for the commercial Smart Grid

Build-up and as the remedy for PV power intermittency. For low cost production of hydrogen, a hybrid reforming system 720 involving electric input obtained from the PV solar power generator 743 is utilized, in conjunction with the use of a renewable biomass to be processed through a renewable feedstock processor 710. The electrical storage mechanism occurs through the energy consumption of the endothermic chemical reforming reaction, steam generation and the reformate compression that constitutes as much as 50% of the energy input for the process. The storage medium may be biomethanol (methanol) or equivalent liquid fuel to be produced in the energy reactor 730 and stored in the liquid fuel storage 741. The power generator 740 can be operated on demand rather than on the unsteady solar supply. The energy system of FIG. 7 can be implemented in the energy system 600 of FIG. 6.

FIG. 8 shows an CO₂-Enhanced Oil Recovery (EOR) system provided in an exemplary embodiment. As the price of energy continues to rise, the interest in oil and gas exploration and production is increasing, relying upon advanced stimulation techniques for improved recovery. As discussed above, a fuel processor or reformer system 820 produces a reformate from an input fuel source, such as natural gas. A compressor 821 compresses the reformate. The hydrogen 822 may be used by the power generator 842 to generate power. The CO₂ gas 823 may be injected into a wellhead 830 of an oil site to stimulate the production of oil.

Since the reformer is installed on site, this system can greatly enhance the operational income. Furthermore, the CO₂ sequestration credit may significantly offset the capital cost of the fuel cell or reformer system, which enhances the financial incentive of the adopting this advanced ZEES system 600 in FIG. 6 for stimulating oil and gas production.

As described above, the embodiment in this application utilizes chemical principles for concurrent power generation and energy conversion. The embodiment eliminates the CO₂ emissions from the power plants and introduces new sources of liquid fuel for transportation. Therefore, this application provides solutions to simultaneously cure the two most serious problems in the current energy industry: petro-fuel shortage and global warming due to greenhouse gas (GHG) emissions. In the embodiments, the greenhouse gas is retained and applied for the production of high valued fuels. This invention may be applied equally to renewable feedstock and other feedstock including coal and solid hydrocarbon fuels.

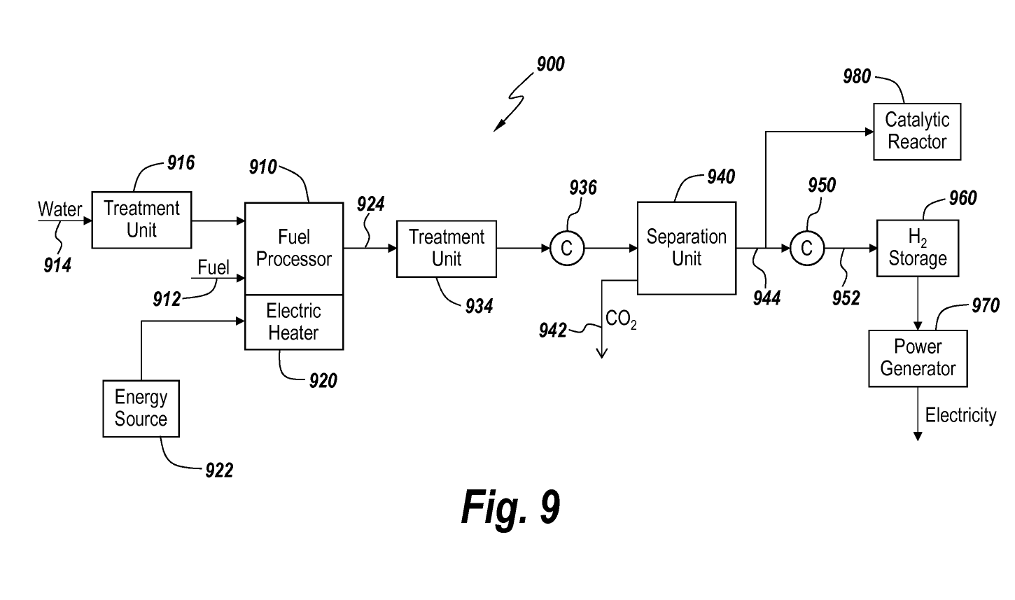

The present invention also contemplates the use of a fuel processor, such as a reformer, that employs a heating unit, such as an electric heater, to provide heating energy for the endothermic needs of the reformer. FIG. 9 is a schematic diagram of another embodiment of the energy system according to the teachings of the present invention. The illustrated energy system 900 can be any type of energy system, and preferably is an environmentally benign, zero or low emission energy system. The energy system can be any size system having any desired power or hydrogen generating capacity or rating. As used herein, the term zero or low emission is intended to include a supply station that has carbon emissions (including CO, CO₂ and CₓH_{y} species) that are 50% less than the carbon content of the hydrocarbon fuel being dispensed or consumed at the system or site, preferably below 25%, and most preferably close to or equal to 0%. Although illustrated with multiple different stages and components, the system can have any selected number of components and arrangements thereof. The illustrated arrangement is merely illustrative and is not intended to be construed in a limiting sense. The description of units and components previously described need not be reproduced below.

The illustrated energy system 900 can include any suitable number or variety of components. The energy system can include a fuel processor 910 that employs a heating unit, such as an electric heating apparatus 920, for supplying thermal energy to the fuel processor. The electric heating apparatus 920 can be an internally and/or externally mounted heater that is sized and configured for providing thermal energy sufficient to accommodate the endothermic thermal needs of the fuel processor.

The fuel processor 910 can be positioned and configured to receive any type or number of inputs (e.g., air, fuel, water, etc.) depending upon the size or type of reforming apparatus. According to one practice, the fuel processor 910 is supplied with a hydrocarbon fuel 912 from a suitable fuel source (not shown), such as described above in connection with the other embodiments of the energy system. If the fuel processor is a reformer, then the reformer can also be supplied with a reforming agent 914, such as water from a suitable source (not shown). The reforming agent can pass through an optional treatment apparatus or unit 916 for treating the reforming agent prior to being supplied to the fuel processor 910. The type of reforming agent treatment or processing unit can be selected depending upon the type of reforming agent used and/or the type of fuel processor. If the reforming agent is water, the treatment unit can comprise for example a deionizer for deionizing the water, a reverse osmosis device, or a vaporizer for vaporizing the water.

According to one practice, the illustrated fuel processor 910 can be any suitable type of fuel reforming apparatus, such as a reformer, and preferably is a steam reformer. Other types of reformers are know in the art, such as auto thermal reformers and the like. The reformer is adapted to receive the hydrocarbon fuel 912 and the reforming agent 914. Those of ordinary skill will readily recognize that the water can be supplied to the reformer as steam. The reformer employs a catalyst material to promote the reformation of the hydrocarbon fuel into simpler reaction species. For example, the hydrocarbon fuel can be catalytically reformed into an output medium or reformate 924, also referred to as syngas, having a mixture of one or more of S, H₂O, H₂, CO, and CO₂. The illustrated reformer reforms the fuel in the presence of the reforming agent to produce a relatively pure fuel stock. Another example of a reformer suitable for use in the illustrated energy supply system 900 is a plate type reformer that is described in U.S. Pat. No. 5,858,314, the contents of which are herein incorporated by reference. According to one practice, a plate-type compact reformer can be employed in the system, although those of ordinary skill will recognize that other types of reformers, including conventional type reactant bed and cylindrical reformers, can be employed.

The heat necessary for the reforming process can be supplied by the electric heating apparatus 920. The electric heating apparatus can comprise one or more heating elements disposed within or external to the reformer. The heating apparatus can also be combined if desired with other types of heating apparatuses, such as combustors, fuel cells and other known types of heat generating apparatus. The heat can be supplied to the reformer by radiation, conduction or convection. The electric heating apparatus 920 can include any selected structure for interfacing with the fuel processor 910 in order to control, adjust or regulate the temperature thereof, or additionally another component of the energy system 900.

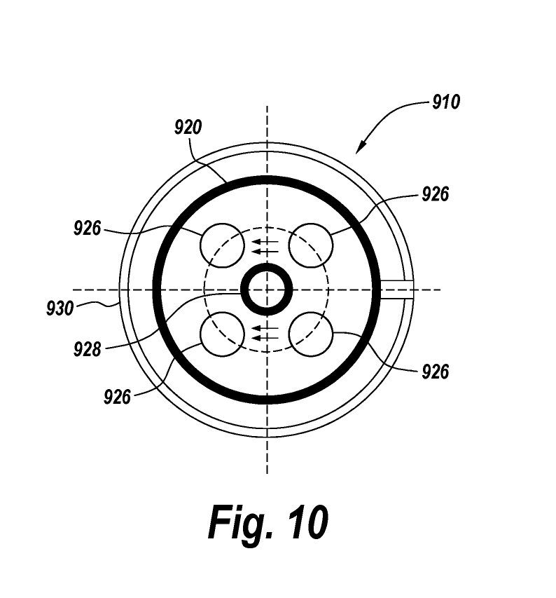

According to one embodiment, the fuel processor 910 can employ an internally mounted electric heating apparatus 920 as shown for example in FIG. 10. The fuel processor 910 includes an outer housing 930 that houses the main processing components. The housing can provide connections for the required inputs, such as water or steam, fuel, and electricity. The illustrated embodiment employs one or more internally mounting heating devices, although the heating devices can also be externally mounted. The heating devices can include the electric heating apparatus 920 disposed about one or more reforming units 926. If desired, a centrally located heating device 928 can also be used. The centrally located heating device can be an electric heating apparatus 920 or any other type of heating device, such as a gas or combustion type heater. Those of ordinary skill in the art will readily recognize that any suitable number or type of reforming units can be employed and the units can be arranged in any selected manner within the housing 930. The present invention also contemplates the use of different types of reforming units within the housing, and the reforming units can also be supplemented with other processing or power generating devices, such as fuel cells.

The power or energy consumed by the electric heating apparatus 920 can be supplied by an energy source 922. The energy source can employ renewable and/or nonrenewable energy sources as desired. As used herein, the term “renewable energy source” refers to any energy source with a natural replenishment rate that augments its own stock (or biomass) at a non-negligible rate. Renewable resources are generally capable of being replenished at least as fast as the renewable resource is used, although this need not be the case. Renewable sources include, but are not limited to, wind, solar energy, geothermal energy, biomass, waste, wave energy and hydro energy. In contrast, nonrenewable energy sources draw on finite resources that will eventually dwindle.

The reformate 924 generated by the fuel processor 910, which can include a mixture of one or more of S, H₂O, H₂, CO, and CO₂, can be supplied to an optional treatment unit 934 that can include a desulfurization unit for removing sulfur or other impurities from the reformate. The treated reformate can then be supplied to an optional compressor 936 for compressing the reformate prior to introduction to a separation or processing unit 940. The separation unit 940 can comprise one or more stages or units adapted to remove, separate and/or isolate individually one or more of the water, hydrogen and/or carbon dioxide from the reformate. Following removal or separation of the steam from the reformer output medium, such as by condensation techniques and associated apparatus, hydrogen can also be extracted from the stream by the separation unit 940, and the remaining carbon dioxide 942 can be optionally removed from the separation unit and if desired collected, sequestered or stored in any suitable manner. The gaseous carbon dioxide can also be converted into a liquid form through, for example, a compressor or by employing known cryogenic techniques. The liquid carbon dioxide can be stored locally or transported off site.

The separation unit 940 can be any suitable unit adapted or configured for processing or separating one or more components from the reformate or output medium produced by the reformer. The separation unit can be configured for separating hydrogen or carbon dioxide from the output medium according to a number of known techniques, including but not limited to chemical or physical absorption, adsorption (e.g., PSA devices), shift reactors, high and/or low temperature distillation devices, high pressure liquefaction units, or employ membrane, enzyme, and/or molecular sieve type separation techniques. The shift reactors of the separation unit 940, if employed, enrich the hydrogen content of the stream, and then subsequent apparatus of the separation stage is capable of removing respectively water and carbon gas, such as CO₂ and CO, and sulfur from the output stream.

After exiting the separation unit 940, the hydrogen-rich output medium or gas 944 is provided for further processing as described above and below. Optionally, the carbon dioxide 942 exiting the separation unit can be channeled for subsequent use or storage. As used herein, the term “hydrogen rich gas” is intended to include a fluid or gas rich in hydrogen, and may include any number of other types of fluids, gases or gas species, such as residual gases including CO₂, CO, H₂O, and unprocessed or unreformed fuel.

The hydrogen rich gas 944 can then be conveyed to an optional compressor 950 where the hydrogen rich gas is further compressed. The compressed hydrogen 952 can then be conveyed by any suitable fluid conduit to a hydrogen storage unit 960 for storing the hydrogen. The stored hydrogen can then be subsequently used by a power generator 970 for generating electricity. The power generator can be any suitable type of generator configured to generate electricity of power from an input gas, such as hydrogen. Examples of suitable power generators include fuel cells, turbines, engines and the like. Alternatively, the hydrogen rich gas 944 can be supplied directly or indirectly to the power generator.

According to another practice, the hydrogen rich gas 944 can be conveyed to a catalytic reactor 980, similar to the reactors described above in connection with the other embodiments, such as the reactors shown and described in FIGS. 2, 3 and 7. The catalytic reactor 980 catalytically converts the hydrogen and carbon species present in the hydrogen rich gas stream into a liquid fuel, such as methanol. The methanol can be further processed as desired.

The present invention is thus directed to an energy system that includes a reformer that employs an electric heating apparatus for providing thermal energy to supply the necessary heat to satisfy the endothermic needs and other thermal and electrical functions of the reforming process. The use of the electric heating apparatus with the reformer thus forms a hybrid reformer system. The reformer can generate a gaseous reformate, which includes H₂, CO₂, and CO, which can be further processed to remove the hydrogen from the reformate. The hydrogen can be converted, if desired, into a liquid fuel or a liquid chemical by employing a catalytic reactor. The energy system thus converts the gaseous reformate and isolated hydrogen into a liquid (GTL).

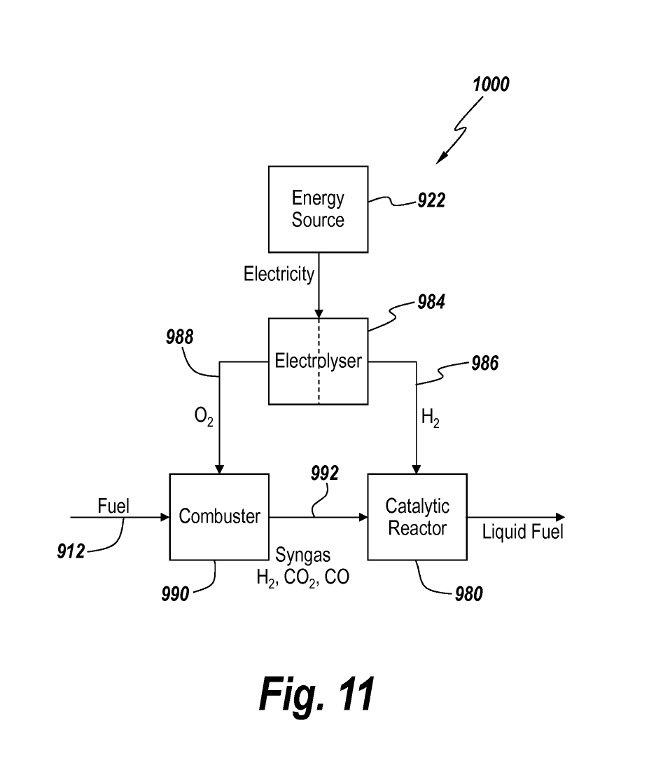

FIG. 11 is a schematic illustration of an energy system 1000 according to another embodiment of the present invention. The energy system of this embodiment employs an electrolyzer for use with other system components to generate a liquid fuel according to the teachings of the present invention Like reference numerals are used to depict like components. The illustrated energy system 1000 employs an energy source 922 for generating and supplying electricity to an electrolyzer 984. The energy source alternatively employs a renewable energy source. The electrolyzer 984 when supplied with electricity converts, through known electrochemical reactions, an input supply of water (not shown) into separate streams of hydrogen 986 and oxygen 988. The electrolyzer can have any selected shape or size and any selected number of electrolyzers can be used in the illustrated energy system. The oxygen stream 988 can be conveyed via any suitable conduits to a combustor 990. An input hydrocarbon fuel 912 is also supplied to the combustor 990. The combustor 990 combusts to known techniques the fuel 912 in the presence of the oxygen 988 to produce a combustion exhaust medium or syngas 992 that includes CO₂ and CO. The combustor can have any selected size and shape as would be readily apparent to one of ordinary skill in the art.

The exhaust medium 992 is conveyed to the catalytic reactor 980, which mixes and chemically reacts the hydrogen 986 received from the electrolyzer 984 with the exhaust medium 992 to produce a liquid fuel, such as methanol. The methanol can be stored locally at the production facility or can be transported for storage off site.

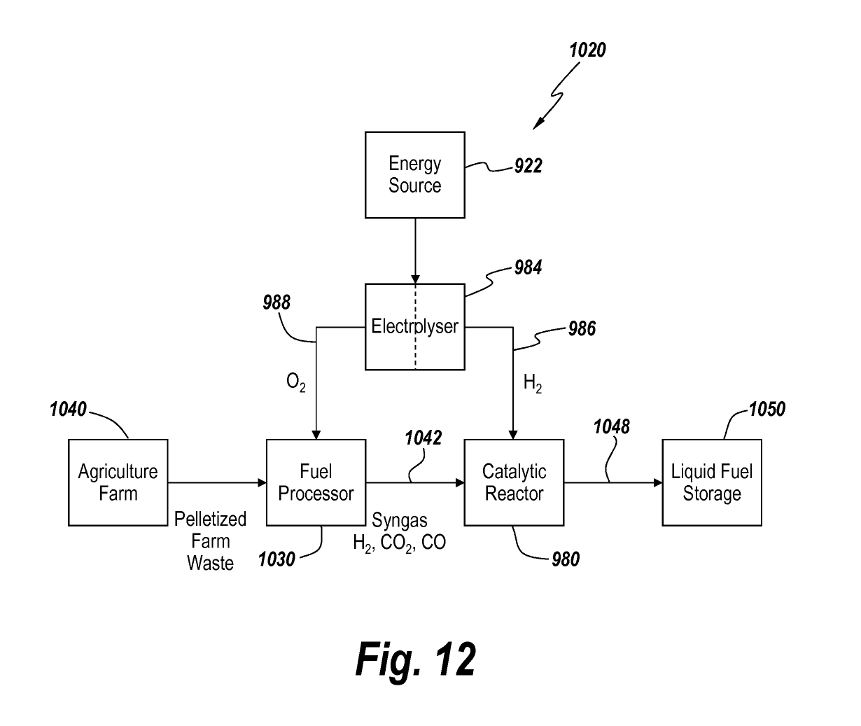

FIG. 12 is a schematic illustration of an energy system 1020 according to yet another embodiment of the present invention. The energy system of this embodiment also employs an electrolyzer with other system components to generate a liquid fuel according to the teachings of the present invention. Like reference numerals are used to depict like components. The illustrated energy system 1020 employs an energy source 922 for generating and supplying electricity to an electrolyzer 984. The energy source preferably employs a renewable energy source. The electrolyzer 984 converts through known electrochemical reactions an input supply of water (not shown) into separate streams of hydrogen 986 and oxygen 988. The electrolyzer can have any selected shape or size and any selected number of electrolyzers can be used in the illustrated energy system. The oxygen stream 988 can be conveyed via any suitable conduits to a fuel processor 1030. The fuel processor 1030 is also adapted to receive solid farm waste as an input fuel from a farm or other agricultural facility 1040. The fuel processor converts the input fuel in the presence of oxygen into a reformate or syngas 1042, which includes H₂, CO₂ and CO, through known techniques. The fuel processor can be a reforming gasifier, a syngas generator, a volatizer and the like.

The exhaust medium 992 is conveyed to the catalytic reactor 980, which mixes and chemically reacts the hydrogen 986 received from the electrolyzer 984 with the reformate 1042 to produce a liquid fuel 1048, such as methanol. The liquid fuel 1048 can be stored locally at the production facility in a suitable storage unit 1050 or can be transported for storage off site.

The liquid fuel produced by the foregoing energy systems of the present invention is easily transportable and has a high commercial value. The fuel processors employed throughout the various embodiments convert an input hydrocarbon fuel, such as methane or natural gas, into a reformate consisting of for example H₂, CO and CO₂. The reformate can be further processed by an energy or catalytic reactor to produce liquid hydrocarbon fuel, such as methanol, DME, gasoline, diesel and other fuels through synthesis applying employing designed catalysts. The catalytic reactor synthesizes the reformate into a liquid fuel, and this process is typically carried out in a temperature range between about 200° C. and about 300° C., and in a pressure range between about 10 atm and about 20 atm. Thus, the electricity assisted reformer followed by use of the catalytic reactor is capable of producing liquid hydrocarbon fuel, such as methanol, DME, gasoline, diesel and others. In essence, the energy systems of the present invention convert an input fuel into energy that can be stored for subsequent use.

As noted above, the most common and economical method for producing hydrogen is by employing a fuel processor, such as a steam methane reformer (SMR). The SMR reforms natural gas in a preferred temperature range between about 700° C. and about 800° C. into a reformate, which ideally consists of about 80% of H₂ and about a 20% mixture of CO and CO₂. In a conventional reformer operation for producing H₂, the endothermic reforming reaction is achieved y using indirect heat from the combustion of natural gas. Thus the CO₂ emission from this type of heating operation cannot be ignored. When combining the reformer heating and the use of compression devices, the energy use can reach up to 50% of the reformer's name plate capacity. Other types of reformers that can be used include autothermal reformers and partial oxidation reformers.

The clean energy systems of the present invention can utilize if desired renewable energy sources to produce renewable hydrogen that serves many clean energy functions. The produced renewable hydrogen can be stored and can be used for various purposes, such as for example as a propulsion fuel for fuel cell vehicles, and as a fuel for clean energy power generation for zero emission battery cars and stationary power needs. The renewable hydrogen can also be liquefied and thus stored as liquid hydrogen. The renewable hydrogen can be made storable by reacting with CO₂ in a methanation process to form renewable gaseous fuel (e.g., methane CH₄). Renewable hydrogen can be made storable by reacting with CO₂ in a GTL process to form renewable liquid fuel (e.g., CH₃OH).

Further, the renewable energy sources are derived from surplus electricity from renewable energy sources, such as a wind power or a solar power facility or any other underutilized power generation equipment.

The clean energy system of the present invention also contemplates the use of an electrolyzer using renewable electricity. The electrolyzer inverts the surplus renewable electricity to renewable hydrogen for storage and converts via known power generators, such as fuel cells, the renewable hydrogen into zero emission type clean electricity. The renewable hydrogen can be stored as pressurized hydrogen in small quantities or as liquefied Hydrogen in large quantities.

According to the energy systems of the present invention, the electricity produced by the raw renewable sources can be converted into different types of fuel and then subsequently stored so as to be available on-demand as a fuel source for commercial power.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover generic and specific features of the invention described herein, and all statements of the scope of the invention which, as a matter of language, might be said to fall therebetween.

Having described the invention, what is claimed as new and desired to be secured by Letters Patent is:

What is claimed is:

1. An energy system, comprising
a fuel processor for receiving a hydrocarbon fuel and for catalytically converting the hydrocarbon fuel into a reformate,
an electric heating apparatus coupled to the fuel processor for providing thermal energy to the fuel processor,
a renewable energy source coupled to the electric heating apparatus for providing power thereto, and
a catalytic reactor for processing the reformate and for converting the reformate into a liquid fuel or a liquid chemical.

2. The energy system of claim 1, wherein the fuel processor comprises a reformer.

3. The energy system of claim 2, wherein the reformer is a partial oxidation reformer, an autothermal reformer, a steam methane reformer, or a steam reformer.

4. The energy system of claim 1, wherein the electric heating apparatus is disposed within the fuel processor.

5. The energy system of claim 1, wherein the electric heating apparatus is disposed external to the fuel processor.

6. The energy system of claim 1, wherein the energy source is a renewable solar or wind energy source.

7. The energy system of claim 1, further comprising a separation unit coupled to the fuel processor for separating hydrogen from the reformate.

8. The energy system of claim 7, further comprising
a compressor coupled to the separation unit for compressing the hydrogen separated from the reformate by the separation unit, and
a storage element for storing the hydrogen compressed by the compressor.

9. The energy system of claim 1, wherein the reformate includes at least H₂, CO, CO₂ and H₂O.

10. The energy system of claim 1, wherein the fuel processor comprises a housing defining a chamber, and a plurality of reformers disposed within the housing, wherein the electric heating apparatus is mounted within the chamber for supplying thermal energy to the plurality of reformers.

11. The energy system of claim 10, wherein the electric heating apparatus has a cylindrical shape and is disposed about the plurality of reformers.

12. The energy system of claim 10, further comprising a second heating apparatus disposed within the chamber for supplying thermal energy to the plurality of reformers.

13. The energy system of claim 1, wherein the system is constructed as a hydrogen fueling station, and the system further comprising
a hydrogen liquefier for liquefying hydrogen produced by the fuel processor,
a storage element for storing the liquefied hydrogen,
a vaporizer,
a GH₂ holding tank,
an ionic compressor,
a GH₂ precooler, and
a hydrogen dispenser.

14. The energy system of claim 1, wherein the hydrocarbon fuel is natural gas only for startup operation, and once the system reaches a steady state operation, a portion of the H₂ generated by the fuel processor can be recycled to replace the natural gas as an input fuel, so as to form a zero emission system.

15. The energy system of claim 1, wherein the system generates CO₂ and utilizes the CO₂, which can be pressurized or stored in a liquefied state, to fulfill the renewable function in energy or power system operation.

16. The energy system of claim 1, wherein the system can practice steam recycle to enhance the thermal efficiency of a co-production process, which is to recycle steam production and utilization between power production and reforming process

17. The energy system of claim 1, wherein the system converts renewable raw electricity to a form suitable for storage to become dispatchable as on demand regulated commercial power, such as on peak or average renewable power rating.

18. The energy system of claim 1, wherein the fuel processor produces a reformate having at least H₂, CO₂, and CO, and is further processed into two separate streams, including a stream of relatively pure H₂ and a stream of CO₂, wherein the stream of H₂ is employed for zero emission power generation and the stream of CO₂ is applied for CO₂ chemical and commercial usage.

19. An method for producing a liquid fuel, comprising, catalytically converting a hydrocarbon fuel into a reformate with a fuel processor, providing thermal energy to the fuel processor by an electric heating apparatus, providing power to the electric heating apparatus by an energy source, and processing the reformate and converting the reformate into a liquid fuel with a catalytic reactor.

20. The method of claim 19, wherein the electric heating apparatus is disposed within the fuel processor.

21. The method of claim 19, wherein the electric heating apparatus is disposed external to the fuel processor.

22. An energy system, comprising,
an energy source for providing electricity,
an electrolyzer positioned so as to receive the electricity from the energy source and water from a water source, for converting the water into a hydrogen stream and an oxygen stream,
a combustor for combusting a fuel received from a fuel source and the oxygen stream received from the electrolyzer and for generating an exhaust, and
a catalytic reactor for processing the exhaust from the combustor and the hydrogen stream from the electrolyzer for producing a liquid fuel or a liquid chemical.

23. The energy system of claim 22, wherein the energy source is a renewable solar energy source or a renewable wind energy source.

24. The energy system of claim 22, wherein the liquid fuel or the liquid chemical can be used for electric energy storage with no strict constraints in quantity or storage shelf life.

25. The energy system of claim 22, wherein the liquid chemical can be used in the form of plastic from methanol to olefins plastics by employing an MTO process with zeolite H-SAPO-34, and which can be disposed as HC solid waste without releasing CO₂ to the atmosphere.
